(19) United States
(12) Patent Application Publication
(10) Pub. No.: US 2010/0280351 A1
(43) Pub. Date: Nov. 4, 2010

(54) APPARATUS AND METHOD FOR ASSESSING ROTATIONAL BONE KINEMATICS

(76) Inventor: Jae Hyuk Shin, Seoul (KR)

Correspondence Address: HAMRE, SCHUMANN, MUELLER & LARSON, P.C., P.O. BOX 2902, MINNEAPOLIS, MN, 55402-0902, US

(21) Appl. No.: 12/678,605

(22) PCT Filed: Sep. 19, 2008
(86) PCT No.: PCT/KR08/05563

§ 371 Date: Jul. 1, 2010

(30) Foreign Application Priority Data
Sep. 20, 2007 (KR) 10-2007-0095999

(51) Int. Cl. A61B 5/05; G06K 9/00

(52) U.S. Cl. 600/407; 382/128

(57) ABSTRACT

Apparatus and method are disclosed for assessing rotational bone kinematics. Instantaneous center of rotation (ICR) of a bone segment is derived from a digitalized image of a bone and it is compared with the normal distribution of an ICR to determine rotational bone kinematics. The apparatus and method may be used to assess kinematic status of a normal bone and pathological bone. They also may be used to design an improved artificial disc to provide an appropriate kinesis.

### TECHNICAL FIELD

The present invention relates to an apparatus and method for assessing a rotational bone kinematics, and more particularly, to an apparatus and method for assessing a rotational bone kinematics, which is capable of assessing the rotational bone kinematics by deriving and comparing a coordinate of instantaneous center of rotation (ICR) of a bone segment with a normal distribution of an ICR of a bone.

### BACKGROUND ART

Bones such as cervical vertebrae, thoracic vertebrae, and lumbar vertebrae are composed by multiple bone segments connected by interposed discs. Discs are composed of outer annulus fibrosus and inner core of nucleus pulposus. Herniation of nucleus pulposus (HNP) is protrusion or extrusion of the disc usually to dorsal direction and compresses the nerves located thereby, which causes pathologic pain.

In HNP, the aim of treatment is decompressing the nerves which are under pathologic compression. A typical treatment aims at removing the disc and bone which compresses the nerves. In cervical spine, removal of the intervertebral disc at anterior column of the spine axis necessitates subsequent reconstruction of the anterior column. Conventional and traditional anterior reconstruction is fusion of the segment by inserting autogenous bone graft or specifically designed spacers such as cages (Abbed K M and Coumans J V. Cervical radiculopathy: pathophysiology, presentation, and clinical evaluation. Neurosurgery 2007; 60S28-34 and Bartolomei J C, Theodore N and Sonntag V K. Adjacent level degeneration after anterior cervical fusion: a clinical review. Neurosurg Clin N Am 2005; 16575-87, v.)

However fusion of spinal segments limits the motion which used to be present; the adjacent vertebral segments are exposed to higher level of stress in order to create original level of motion, which is intended by the patient. This increased stress on the adjacent segments is known to accelerate development of degenerative change at the adjacent segment (adjacent segment disease).

Artificial disc is recently developed prosthesis which is largely intended suggest solution to these problems. After sufficiently decompressing the disc to free the nerve, the inserted artificial disc provides motion to the segment instead of fusing the segment.

Implanting the prosthesis requires post-surgical evaluation. Conventionally, it is assessed whether the artificial disc succeeds in providing motion of the bone; however, it is not assessed whether the artificial disc provides ‘the normal kinesis’ of the bone. Up to now, method or system for assessing whether a bone such as a cervical vertebra is normally kinetic and whether the inserted artificial disc provides a normal kinesis as a normal physiologic bone has not been developed. Accordingly, there is a need for the development of an efficient method or system for assessing the quality of rotational bone kinematics.

### DISCLOSURE OF INVENTION

### Technical Problem

Therefore, the present invention has been made in view of the above problems, and it is an object of the present invention to provide an apparatus for assessing a rotational bone kinematics by deriving and comparing a coordinate of an instantaneous center of rotation (ICR) of a bone segment with a normal distribution of an ICR of a bone.

It is an another object of the present invention to provide a method for assessing a rotational bone kinematics, which is capable of accurately assessing whether or not the kinesis of a bone is normal after an artificial disc is inserted and designing the artificial disc for providing a kinesis closest to that of an original human bone.

### Technical Solution

In accordance with an aspect of the present invention, the above and other objects can be accomplished by the provision of an apparatus for assessing a rotational bone kinematics, the apparatus including: an image acquiring unit which acquires digitalized images of a bone including at least two bone segments in a flexion position and an extension position; a normal distribution database which stores normal distribution data of instantaneous centers of rotation (ICRs) of the bone segments; a rotational bone kinematics assessing unit which receives digital image data of the bone from the image acquiring unit, derives the ICRs of the bone segments, compares the derived ICRs with normal distributions of the ICRs read from the normal distribution database, and outputs an assessed result; and a display unit which displays the acquired images and the assessed result.

According to another aspect of the present invention, there is provided a method for assessing a rotational bone kinematics, the method including: acquiring digitalized images of a bone including at least two bone segments in a flexion position and an extension position; deriving instantaneous centers of rotation (ICRs) of the bone segments from the digitalized image data in the flexion position and the extension position; collecting data on the ICRs of normal distributions for the ICRs of the bone and establishing a normal distribution database of the ICRs of the bone; and reading the normal distribution data from the normal distribution database, comparing the normal distribution data with the derived ICRs, and assessing the rotational bone kinematics depending on whether the ICRs are in the respective normal distributions.

### ADVANTAGEOUS EFFECTS

According to an apparatus for assessing a rotational bone kinematics of the present invention, it is possible to obtain a distribution of instantaneous centers of rotation (ICRs) of a normal cervical vertebra and determine whether the ICRs of bone segments of a bone to be assessed is in the respective normal distribution ranges so as to determine whether the rotational bone kinematics is normal. According to the apparatus and method of the present invention, it is possible to measure the accuracy of motion that an artificial disc creates. Preoperative motion and postoperative motion could be assessed whether physiologic or pathologic, respectively. Not only pre-operative-postoperative difference could be measured, but also each device could be compared with another device regarding accuracy of the ICR.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a schematic view of an apparatus for assessing a rotational bone kinematics according to an embodiment of the present invention;

FIG. 2 is a detailed block diagram showing a rotational bone kinematics assessing unit of the apparatus for assessing the rotational bone kinematics shown in FIG. 1;

FIG. 3 is a view illustrating a method for setting coordinates of vertebrae according to the present invention;

FIG. 4 is a view illustrating a method for obtaining a coordinate of a second cervical vertebra (C2) according to the present invention;

FIG. 5 is a view illustrating a method for deriving an instantaneous center of rotation

(ICR) of a cervical disc;

FIG. 6 is a view showing the ICR of the cervical disc and a normal distribution of the ICR having a confidence level of 95%;

FIG. 7 is a view illustrating a process of representing the ICR of the cervical disc by the coordinates;

FIG. 8 is a view showing an example of the normal distributions of the ICRs of bone segments of the cervical vertebra and the assessed ICRs;

FIG. 9 is a flowchart illustrating a method for assessing a rotational bone kinematics according to an embodiment of the present invention;

FIG. 10 is a flowchart illustrating a process of deriving the ICRs of bone segments; and

FIG. 11 is a flowchart illustrating a process of storing the normal distribution of the ICRs of the cervical disc having a normal status in a database.

### MODE FOR THE INVENTION

Hereinafter, the present invention will be described with reference to the accompanying drawings.

An apparatus for assessing a rotational bone kinematics according to an embodiment of the present invention photographs an image of a bone including a plurality of bone segments, derives instantaneous centers of rotation (ICRs) of the bone segments from the digitalized image of the bone, compares the ICRs with normal distributions of the ICRs of the bone, and assesses the rotational bone kinematics. This apparatus may be used to assess the rotational bone kinematics including the plurality of bone segments, such as a cervical vertebra, a thoracic vertebra, or a lumbar vertebra. According to the apparatus of the present invention, it is possible to accurately assess whether the bone of a person to be inspected provides a normal kinesis or whether the artificial disc of a patient who undergoes an operation for inserting the artificial disc provides a normal kinesis and it is possible to contribute to improving quality of rotation profile in newly developed artificial discs.

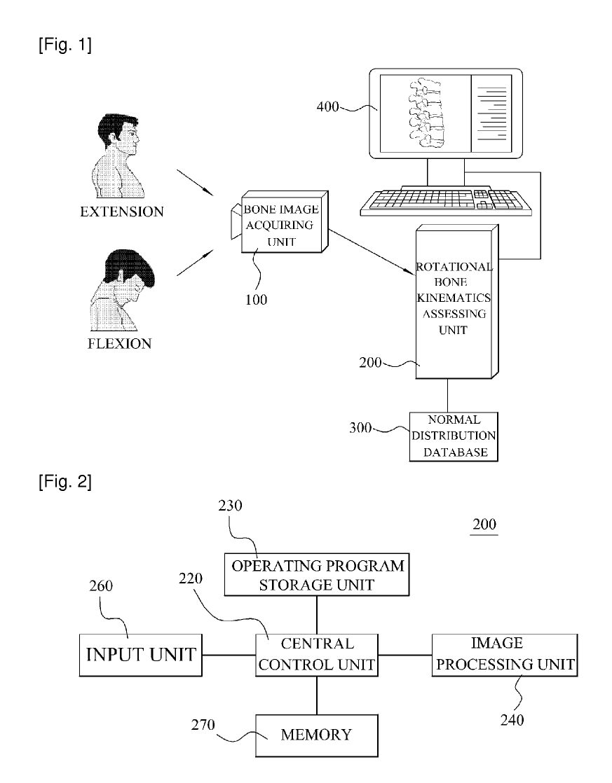

FIG. 1 is a schematic view of an apparatus for assessing a rotational bone kinematics according to an embodiment of the present invention. FIG. 2 is a detailed block diagram showing a rotational bone kinematics assessing unit of the apparatus for assessing the rotational bone kinematics.

Referring to FIG. 1, the apparatus for assessing the rotational bone kinematics according to the embodiment of the present invention includes an image acquiring unit 100 which acquires digitalized images of a bone including at least two bone segments in flexion and extension positions; a normal distribution database 300 which stores normal distribution data of the ICRs of the bone segments; a rotational bone kinematics assessing unit 200 which receives the digital image data of the bone received from the bone image acquiring unit, derives the ICRs of the bone segments, compares the derived ICRs with the normal distributions of the ICRs read from the normal distribution database, and outputs the assessed result; and a display unit 400 which displays the acquired images and the assessed results.

FIG. 1 shows an example of assessing the cervical vertebra. If the neck of the person to be inspected is stretched, the cervical vertebra is in the extension position and, if the neck of the person to be inspected is flexed, the cervical vertebra is in the flexion position. The images of the bone in the extension position and the flexion position are acquired. The image acquiring unit 100 may include any device for photographing the bone, such as an X-ray imaging device, a sonography device, an X-ray computed tomography (CT) device or a magnetic resonance imaging (MRI) device. The image acquiring unit 100 converts the acquired images of the bone into digitalized signals by a digitalizing system, and transmits the digitalized signals to the rotational bone kinematics assessing unit 200. The rotational bone kinematics assessing unit 200 can be embodied by an image processing apparatus, such as a computer, which processes and reconstructs the digitalized image signals transmitted from the image acquiring unit 100 so as to reconstruct final images.

Although the image acquiring unit 100 and the rotational bone kinematics assessing unit 200 may be directly connected to each other, they are mostly connected to each other via any interface (not shown) such as a wired/wireless communication line of a hospital.

The normal distribution database 300 stores the data on the corresponding normal distribution ranges of the ICRs of the bone segments of a certain bone (e.g., a cervical vertebra, a thoracic vertebra, or a lumbar vertebra). The data on the ICRs of various bones stored in the normal distribution database represents information for normal population which is obtained and stored by a method for photographing the bones of people from various ages and various physique conditions without a pathological status and by obtaining the ICRs of the bone segments.

The rotational bone kinematics assessing unit 200 includes a central control unit 220 which derives the ICRs of the bone segments of a bone to be assessed and outputs the derived result to an image processing unit; a memory 270 which stores information on the ICRs of the bone derived by the central control unit; the image processing unit 240 which converts the image data of the bone received from the central control unit into an image signal displayed by the display unit 400; an input unit 260 which is connected to the central control unit and receives a command of the user; and an operating program storage unit 230 which stores an operation program of the central control unit and a whole operating program of the apparatus.

The rotational bone kinematics assessing unit 200 may obtain the ICRs on a one-dimensional coordinate system, a two-dimensional coordinate system or a three-dimensional coordinate system. A technology of three-dimensionally reconfiguring a medical image is widely known in the technical field of the present invention and includes a series of conversion processes for reconstructing a three-dimensional bone morphology, and utilizes the acquired multi-dimensional image data. That is, the three-dimensional image is reconstructed by consecutively summing up the two-dimensional cross-sectional images by predetermined intervals.

In the central control unit 220, two or more points are selected either in extension position or in flexion position from images received from the bone image acquiring unit 100; their corresponding points in counter-position (flexion versus extension) are traced by calculation. The corresponding pair-points are connected with straight lines; perpendicular bisectors of these straight lines intersect at body of the lower vertebra in sagittal image, which designates the ICR of the bone segment. This process is repeated with respect to the bone segments such that the ICRs of each bone segment are obtained.

At this time, the coordinate of the ICR can be obtained by forming a rectangle in each bone segment on the basis of a inferior posterior point, dividing the rectangle in a lattice shape having a predetermined size in vertical and horizontal directions so as to form a coordinate system, and obtaining ratios of horizontal and vertical positions of the ICR to a total length in this coordinate system. Since the coordinate position of an individual is represented relative to the total length of the vertebra of the individual, it is important that the vertical and horizontal lengths of the bone (the cervical vertebra, the thoracic vertebra, or the lumbar vertebra) are measured. Since the cervical vertebra does not necessarily form a rectangle when viewed laterally, an error may occur in the measurement. In particular, since an osteophyte which is a degenerative variation frequently occurs in the inferior posterior point, it is difficult to be consecutively accurate in selecting the points according to blurring or the exact location by the degenerative condition (the degree that the osteophyte occurs) of the individual. Accordingly, in the present invention, the coordinate may be set as follows.

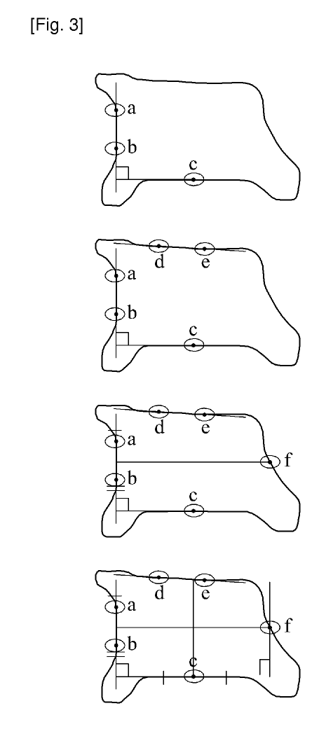

Now, a method for forming a rectangular coordinate system according to the present invention will be described with reference to FIG. 3. First, as shown in FIG. 3, two points a and b are placed—by a person—in a posterior side (the left side of the vertebra in the drawing) creating a initial straight line. The inferior surface forms an upward-convex shape and the apex c is placed—by a person; extending a line which is perpendicular to the straight line of the posterior side—by digital calculation—creates a coordinate system. Next, two points d and e are placed on an superior side of the vertebra—by a person; extending the line—by digital calculation—creates a trapezoid shape. A point f, in which the perpendicular bisector of the posterior side (on the basis of the inferior side) meets a anterior side, is placed and the length of the segment of the line is referred to as the horizontal length of the vertebra. This contact point of f could be either determined by computer or by a person: computer could identify a point where the extending line meets the cortical bone density in computed radiographic density; this point could be manually pointed by a person; or computer initially guides a point and a person may confirm the location. From the point f, a straight line perpendicular to the inferior side is drawn; a line is drawn as the perpendicular bisector of the lower side until it meets the superior side. The length of the segment of the line is referred to as the vertical length of the vertebra. Accordingly, the lattice-shaped coordinate system can be set.

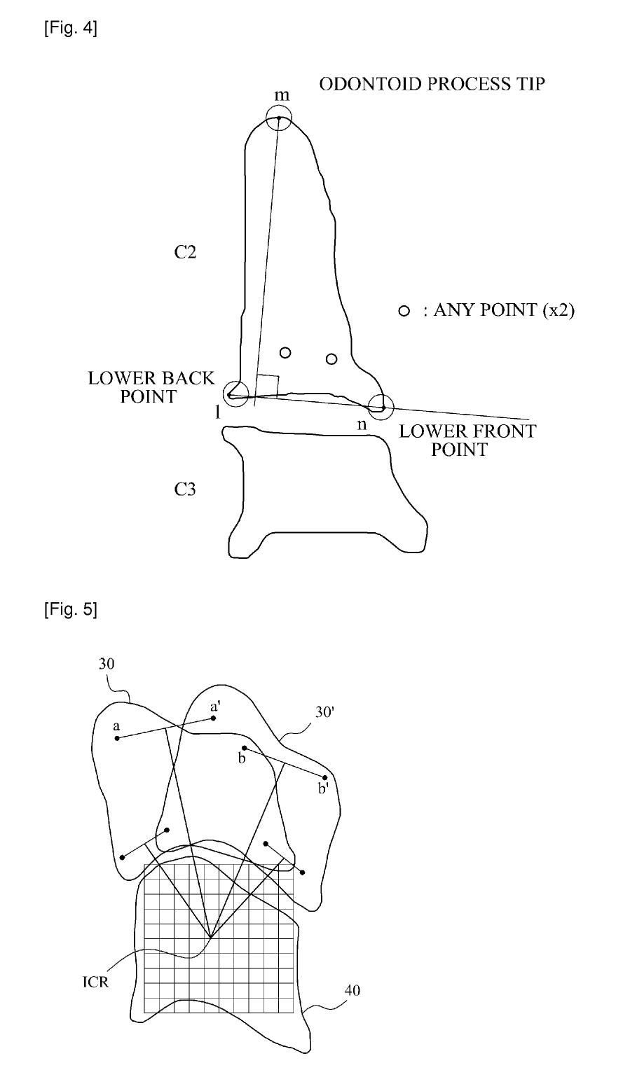

Meanwhile, since a second cervical vertebra C2 has a unique shape, the coordinate system may be set by another method. As shown in FIG. 4, since the ICR of the second cervical vertebra C2 is located within the third cervical vertebra C3, the second cervical vertebra C2 does not need to contain an ICR. However, obtaining the ICR necessitates initial pointing of the rotating points, which is beginning for drawing the perpendicular bisector and for obtaining the ICR; the points of the second cervical vertebra C2 should be represented by coordinates. Accordingly, when a inferior anterior point n and a inferior posterior point 1 of the vertebra are placed on the second cervical vertebra C2, a coordinate system having a [symbol] shape is automatically formed; the horizontal coordinate of any rotating point could be computed on basis of this coordinate system. A point m is placed on an odontoid process tip of the second cervical vertebra C2; distance between this point and the inferior side of the second cervical vertebra C2 (a straight line passing through the inferior anterior point and the inferior posterior point) is obtained. The vertical coordinates of any rotating points are calculated by ratio of distances—from the rotating point and from the odontoid process tip- to the inferior side of the second vertebra C2.

Meanwhile, since a second cervical vertebra C2 has a unique shape, the coordinate system may be set by another method. As shown in FIG. 4, since the ICR of the second cervical vertebra C2 is located within the third cervical vertebra C3, the second cervical vertebra C2 does not need to contain an ICR. However, obtaining the ICR necessitates initial pointing of the rotating points, which is beginning for drawing the perpendicular bisector and for obtaining the ICR; the points of the second cervical vertebra C2 should be represented by coordinates. Accordingly, when a inferior anterior point n and a inferior posterior point 1 of the vertebra are placed on the second cervical vertebra C2, a coordinate system having a [symbol] shape is automatically formed; the horizontal coordinate of any rotating point could be computed on basis of this coordinate system. A point m is placed on an odontoid process tip of the second cervical vertebra C2; distance between this point and the inferior side of the second cervical vertebra C2 (a straight line passing through the inferior anterior point and the inferior posterior point) is obtained. The vertical coordinates of any rotating points are calculated by ratio of distances—from the rotating point and from the odontoid process tip- to the inferior side of the second vertebra C2.

The rotational bone kinematics assessing unit 200 determines whether the coordinates of the ICRs of the bone segments are in the respective normal distribution ranges of the bone stored in the normal distribution database. If the coordinates are in the respective normal distribution ranges, it is determined that the rotational bone kinematics is normal and, if the coordinates are outside the normal distribution range, it is determined that the rotational bone kinematics is abnormal.

The apparatus of the embodiment of the present invention may be implemented by an apparatus for assessing whether a bone into which an artificial disc is inserted provides a kinesis similar to that of the bone of the human being, in a patient who undergoes an artificial disc inserting operation. In such an apparatus, the image acquiring unit 100 extracts and digitalizes the image of the artificial bone after the artificial disc is inserted and transmits it to the rotational bone kinematics assessing unit 200. The rotational bone kinematics assessing unit 200 derives the coordinates of the ICRs of the bone segments from the image of the bone received from the image acquiring unit 100, compares them with the normal distributions of the ICRs of the bone, and assesses whether the artificial disc provides the kinesis of the normal bone.

Next, the operation of the apparatus of the present invention will be described.

The data on the ICRs of the bones of normal persons is stored in the normal distribution database 300. The image acquiring unit 100 photographs the kinesis of the cervical vertebra of the normal person, that is, the image of the flexion position in which the neck of the person is flexed forward and the image of the extension position in which the neck of the person leans backward, and converts the photographed images into digital data.

FIG. 5 is a view showing a third cervical vertebra 30 in the flexion position and a third cervical vertebra 30′ in the extension position. The process of deriving the ICRs of the bone segments of the cervical vertebrae from a fourth cervical vertebra to a seventh cervical vertebra is performed by the same method.

The image acquiring unit 100 converts the image data of the cervical vertebra in the flexion and the extension positions into the digital data and transmits the digital data to the rotational bone kinematics assessing unit 200 and more particularly the central control unit 220.

The central control unit 220 derives the ICR of the cervical vertebra by the program stored in the operating program storage unit 230 and outputs current image data of the bone to the image processing unit 240. Thus, the image processing unit 240 processes the image signal and displays the processed image signal via the display unit 400, that is, the monitor. The process of deriving the ICR of the cervical vertebra by the central control unit 220 may be implemented by a computer program. The operating program storage unit 230 is connected to the central control unit 220 and stores the program for operating the system and the program for performing the present invention.

With respect to the ICR, as shown in FIG. 5, the image of the third cervical vertebra 30 in the flexion position and the image of the third cervical vertebra 30′ in the extension position are overlapped with each other and two points (a, b) and (a′, b′) located at specific positions of the cervical vertebrae 30 and 30′ are selected. That is, two points a and b of the image of the third cervical vertebra 30 in the flexion position are selected and two points a′ and b′ of the image of the third cervical vertebra 30′ in the extension position are selected. The selection of these points may be performed by selecting any points located at most suitable positions by the recognition of the image or operating the input unit 260 such as a keyboard or a mouse by the person and selecting any two points located at specific positions while the image displayed on the display unit 400 is directly checked. When any points are selected in the image of the flexion (extension) status, the coordinates of those points in the vertebra are obtained and the coordinates of the points of the image in the extension (flexion) status corresponding thereto are obtained. Since the same points are placed in the image in the flexion position and the image in the extension position, the same coordinate points of the images are computed by the computer. Both these two methods may be used. Although the two points a and b are described in the present embodiment, more than two points may be selected to obtain the ICR in order to improve accuracy.

When the two points are selected, the central control unit 220 connects the two points a and a′ and the two points b and b′ by straight lines and draws a vertical line a-a′ and a vertical line b-b′ from the bisected positions of the straight lines toward a direction of a fourth cervical vertebra 40. A point in which the vertical line a-a′ and the vertical line b-b′ intersect in the fourth cervical vertebra 40 is derived as the ICR and is stored in the normal distribution database 300.

When the image of the flexion position and the image of the extension position overlap with each other, lower vertebras of the two images may not accurately overlap with each other. This is because the conditions at the time of photographing the image of the flexion position and the image of the extension position may be slightly changed. In this case, it is assumed that an upper vertebra is rotated on the basis of a rectangle formed by average coordinates of the vertexes of a rectangle or a trapezoid of the lower vertebra.

Several rotation points may be placed in the rotated superior vertebra. If two points are placed, one ICR is obtained and, if three points are placed, three ICRs are obtained (3C2) because any two points are selected. In this case, the average coordinate of the three ICRs may be obtained as the ICR. Similarly, if n points are placed, nC2 ICRs may be obtained by a repeated operation and an arithmetic average coordinate of the coordinates of x and y axes may be calculated as the coordinate of the ICR.

The above-described process is repeatedly performed with respect to all the vertebrae from the fourth cervical vertebra C4 to the seventh cervical vertebra C7. In order to obtain the normal distributions, this process is repeatedly performed with respect to a plurality of normal persons under various conditions and the respective normal distribution ranges of the ICRs of specific bone segments are derived and stored in the database. The normal distributions of the ICRs which are stored in the normal distribution database 300 may be categorized for comparison. For example, a reliable region including a distribution having a confidence level of 90% based on an average of the normal persons, a reliable region including a distribution having a confidence level of 95% based on the average of the normal persons and a reliable region including a distribution having a confidence level of 99% may be shown.

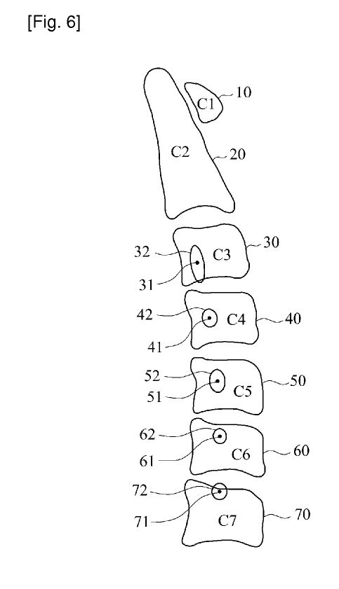

FIG. 6 is a view showing the ICRs of the bone segments of the cervical vertebra and the normal distributions of the ICRs. As shown in FIG. 6, the ICRs of the bone segments C2 to C7 of the cervical vertebra are located in the respective lower bone segments thereof, and the ICR is closer to the front and superior sides of the respective lower cervical vertebra thereof as the bone segment of the cervical vertebra is closer to the lower side. That is, the ICRs 31, 41, 51, 61 and 71 of the second cervical vertebra C2 to the sixth cervical vertebra C6 20, 30, 40, 50 and 60 are located in the respective lower cervical vertebrae thereof. The average points 31, 41, 51, 61 and 71 of the ICRs of the bone segments and the normal distribution ranges 32, 42, 52, 62 and 72 having the confidential level of 95% are described. An experimenter may adjust the boundaries of the normal distributions to 90% or 95%. When the ICRs exist in the distributions, the cervical vertebra provides the normal kinesis in view of the ICR.

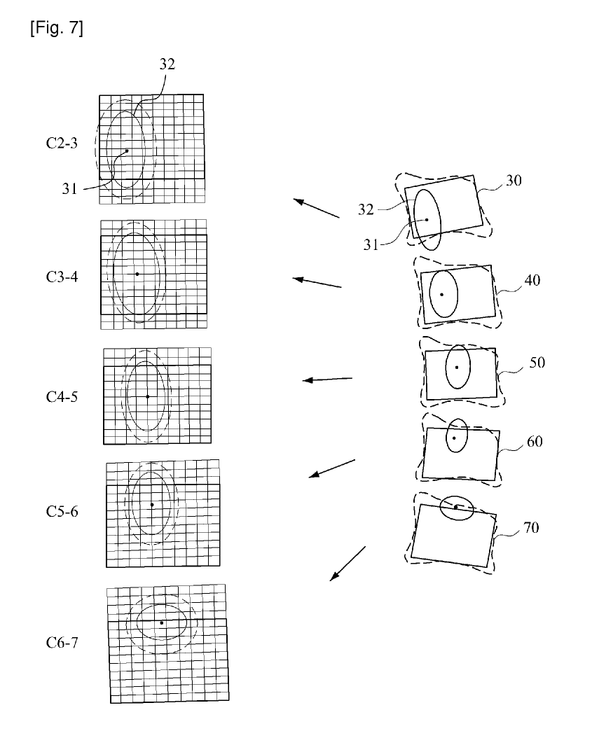

Accordingly, in order to store the normal distributions of the cervical vertebrae for providing the normal kineses, the ICRs of the bone segments of the cervical vertebra need to be represented by coordinates. FIG. 7 is a view showing a process of determining the ICRs of the bone segments of the cervical vertebra on a two-dimensional coordinate system. As shown in FIG. 7, a rectangle is formed on the basis of the inferior posterior point of the lower bone segment. In more detail, four vertexes of each of the bone segments are obtained from the image of the extension position, four vertexes of each of the bone segments are obtained from the image of the flexion position, and the rectangle configured by the vertexes is used as the coordinate system. Subsequently, as shown in the left side of FIG. 7, the obtained rectangle is divided into portions having a predetermined size in the horizontal and vertical directions so as to obtain the coordinate system. In FIG. 7, a dotted line denotes squared paper which becomes the background when all operations are manually performed and a solid line denotes the model of a cervical vertebra which is virtually set. The solid line denotes the region in which, when the ICRs of several persons are standardized, the ICRs of 95% of the persons are included. A dotted ellipse located outside a solid ellipse represents a range which is allowable in consideration of a measurement error.

For example, a rectangle is formed in the third cervical vertebra 30 and is divided by setting the total horizontal and vertical lengths to 100, ratios of the horizontal and vertical positions of the ICR 31 to the total length (e.g., horizontal position: 25/100, vertical position: 65:100) are obtained, and 0.25 and 0.35 are respectively obtained as the X-axis and the Y-axis coordinates. The coordinates of the ICRs of the bone segments of the plurality of normal persons are obtained under the various conditions and the normal distribution 32 of the ICR is stored in the database.

The rotational bone kinematics assessing unit 200 assesses the kinematic status of a specific bone using the normal distribution data stored in the normal distribution database 300. The central control unit 220 of the rotational bone kinematics assessing unit 200 receives the digitalized image data of the bone from the image acquiring unit 100, derives the ICRs of the bone segments by the same method as the above description associated with the establishment of the normal distribution database, and stores the ICRs in the memory 270. Thereafter, the normal distribution data of the ICRs of the bone to be compared is read from the normal distribution database 300.

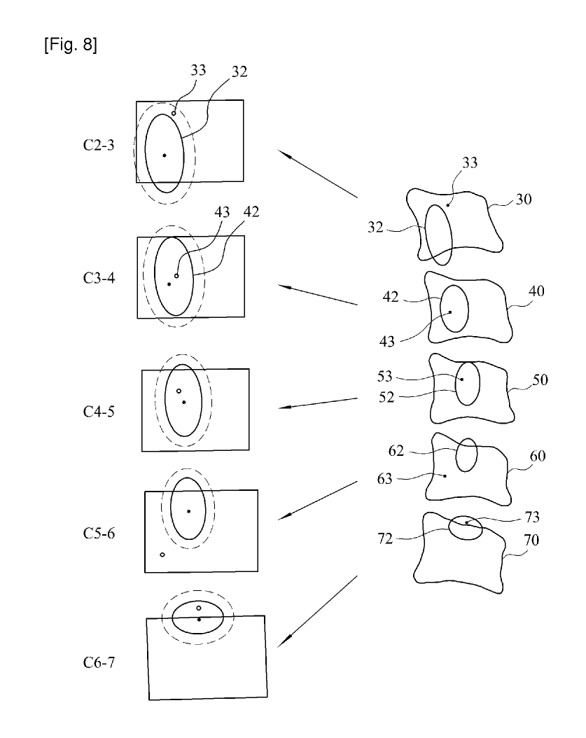

The central control unit 220 compares the data on the ICRs of the bone segments of the bone to be assessed, which is stored in the memory 270, with the normal distribution data, determines that the rotational bone kinematics is normal if the ICRs of the patient are in the respective normal distribution ranges, and determines that the rotational bone kinematics is abnormal if the ICRs of the patient are outside the respective normal distribution ranges. That is, as shown in FIG. 8, if the ICR 33 of the second cervical vertebra is not in the normal distribution range 32 of the third cervical vertebra 30, it is determined that the kinesis of the artificial disc is abnormal and, if the ICR 43 of the third cervical vertebra 30 is in the normal distribution range 42 of the fourth cervical vertebra 40, it is determined that the kinesis of the artificial disc is normal. The kineses of the bone segments are individually assessed.

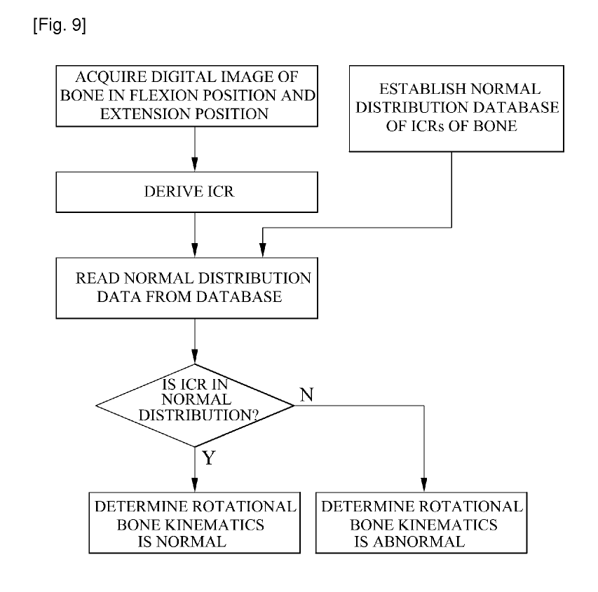

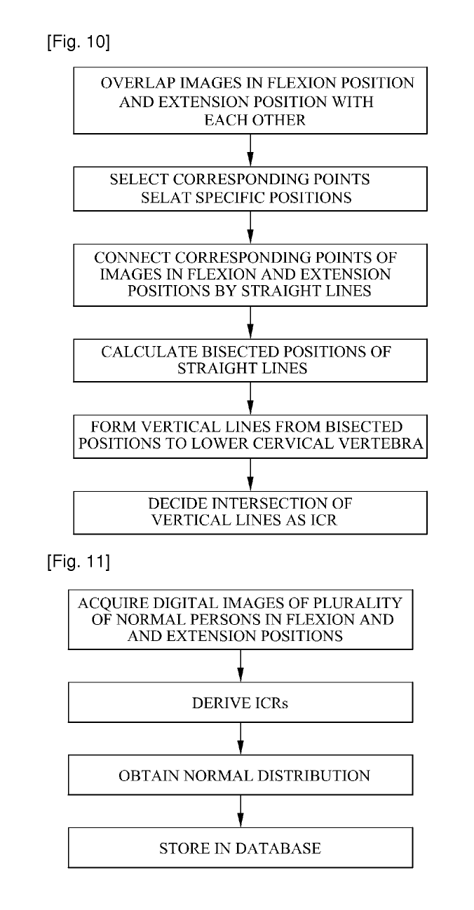

Another aspect of the present invention relates to a method for assessing a rotational bone kinematics. FIG. 9 is a flowchart illustrating a method for assessing a rotational bone kinematics according to an embodiment of the present invention, FIG. 10 is a flowchart illustrating a process of deriving the ICRs of the bone segments, and FIG. 11 is a flowchart illustrating a process of storing the normal distributions of the ICRs of the cervical disc having a normal status in a database.

Referring to FIG. 9, when the kinematic status is assessed by the method of the present invention, first, the digitalized images of the bone including a plurality of bone segments in the flexion position and the extension position are acquired and the ICRs of the bone segments are derived from the digitalized image data of the flexion position and the extension position. Simultaneously with or before this step, the data on the bone segments configuring a bone, such as a cervical vertebra, of each of a plurality of normal persons is collected under various conditions and a normal distribution database of the ICRs is established. In order to assess the kinematic status of a specific bone, normal distribution data of the bone to be assessed is read from the normal distribution database and is compared with the ICR of each of the bone segments of the bone of a patient to be assessed, and the rotational bone kinematics is assessed depending on whether the ICR is in the normal distribution. If the derived ICR is in the normal distribution, it is assumed that the rotational bone kinematics is normal and, if the derived ICR is outside the normal distribution, it is assumed that the rotational bone kinematics is abnormal. The digital images and the assessed result of the bone acquired in the present invention may be displayed on the display unit such as a computer monitor.

Referring to FIG. 10, in the process of deriving the ICR of each of the bone segments, the images of the bone in the flexion position and the extension position overlap with each other and two specific points corresponding to each other in the images of the bone are selected. Subsequently, the two corresponding points of the images of the flexion position and the extension position are respectively connected by straight lines and bisected positions (coordinate average value) of the straight lines connected between the corresponding points are calculated. The respective vertical lines are drawn from the bisected positions of the straight lines toward the lower bone segment and a position in which the vertical lines intersect each other is decided as the ICR of an superior bone segment and is represented by coordinates. At this time, the number of corresponding points of the image of the extension position and the image of the flexion position may be two or more. The correspondence between the two points of the image of the extension position and the two points of the image of the flexion position should be maintained. Accordingly, the coordinate of the corresponding position is computed using the coordinate of any point which is first placed, such that more accurate corresponding points can be obtained.

Referring to FIG. 11, in the process of establishing the normal distribution database, the normal bones of the plurality of normal persons are photographed in the flexion position and the extension position under various conditions and are converted into digital data, and the digital images in the extension position and the flexion position are acquired. The ICRs of the bone segments are derived from the digitalized images of the plurality of normal persons in the flexion position and the extension position and are represented by the coordinates. The ICRs of the normal bones of the plurality of normal persons are concentrated in a predetermined range on the coordinate system. The ICRs of the normal bones are stored in the normal distribution database as the normal distribution. When the normal distribution database is established, the ICRs of the bone segments may be derived by the same method as the above description.

In the process of assessing the rotational bone kinematics, the coordinates of the ICRs of the bone segments to be assessed are compared with the normal distribution data. If the coordinates of the ICRs of the bone segments to be assessed are in the respective normal distributions, it is determined that the rotational bone kinematics is normal and, if the coordinates of the ICRs of the bone segments to be assessed are outside in the respective normal distributions, it is determined that the rotational bone kinematics is abnormal.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

### INDUSTRIAL APPLICABILITY

According to an apparatus and a method of the present invention, it is possible to determine whether or not the ICRs of a patient are normal before an operation is performed, compare an operation result of the patient with the data before the operation in view of the ICRs, and assess the operation result. In addition, by collectively considering a specific apparatus or operation (e.g., anterior fusion for an artificial disc), it is possible to assess a degree that the total result of the apparatus is close to a normal status. For example, by collectively considering the specific apparatus or operation, a variation in ICR may be determined and a degree that a pathological status is changed to a normal status, a degree that a pathological status is changed to another pathological status or a degree that a normal status is changed to a pathological status may be assessed.

1. An apparatus for assessing a rotational bone kinematics, the apparatus comprising:
an image acquiring unit which acquires digitalized images of a bone including at least two bone segments in a flexion position and an extension position;
a normal distribution database which stores normal distribution data of instantaneous centers of rotation (ICRs) of the bone segments;
a rotational bone kinematics assessing unit which receives digital image data of the bone from the image acquiring unit, derives the ICRs of the bone segments, compares the derived ICRs with normal distributions of the ICRs read from the normal distribution database, and outputs an assessed result; and
a display unit which displays the acquired images and the assessed result.

2. The apparatus according to claim 1, wherein the bone is a cervical vertebra, a thoracic vertebra and a lumber vertebra.

3. The apparatus according to claim 1, wherein the image acquiring unit is an X-ray imaging device, a sonography device, an X-ray computed tomography (CT) device or a magnetic resonance imaging (MRI) device.

4. The apparatus according to claim 1, wherein the normal distribution database includes data on normal distribution ranges of the ICRs of the bone segments of a specific bone.

5. The apparatus according to claim 1, wherein the rotational bone kinematics assessing unit obtains the ICRs on a one-dimensional coordinate system, a two-dimensional coordinate system or a three-dimensional coordinate system.

6. The apparatus according to claim 1, wherein the rotational bone kinematics assessing unit includes:
a central control unit which derives the ICRs of the bone segments of the bone to be assessed and outputs the derived result to an image processing unit;
a memory which stores information on the ICRs of the bone derived by the central control unit;
the image processing unit which converts the image data received from the central control unit into an image signal to be displayed on the display unit;
an input unit which is connected to the central control unit and receives a command of a user; and
an operating program storage unit which stores an operation program of the central control unit and a whole operating program of the apparatus.

7. The apparatus according to claim 6, wherein the central control unit derives an ICR from an intersection of perpendicular bisectors from bisectors of lines, which are connected between two corresponding points in an image of the bone in an extension position and an image of the bone in a flexion position received from the image acquiring unit, to a lower bone segment, obtains the ICRs of the bone segments by repeatedly performing the deriving of the ICR with respect to the bone segments, and calculates ratios of the coordinates of the ICRs to total lengths of the bone segments.

8. The apparatus according to claim 6, wherein the central control unit determines whether the coordinates of the ICRs of the bone segments are in the respective normal distribution ranges of the bone stored in the normal distribution database.

9. The apparatus according to claim 1, wherein:
the image acquiring unit extracts and digitalizes the image of an artificial bone after an artificial disc is inserted, and
the rotational bone kinematics assessing unit drives and compares the coordinate of the ICR of the artificial bone with the ICR of a normal bone and assesses whether the artificial disc provides a normal kinesis.

10. A method for assessing a rotational bone kinematics, the method comprising:
acquiring digitalized images of a bone including at least two bone segments in a flexion position and an extension position;
deriving instantaneous centers of rotation (ICRs) of the bone segments from the digitalized image data in the flexion position and the extension position;
collecting data on the ICRs of normal distributions for the ICRs of the bone and establishing a normal distribution database of the ICRs of the bone; and
reading the normal distribution data from the normal distribution database, comparing the normal distribution data with the derived ICRs, and assessing the rotational bone kinematics depending on whether the ICRs are in the respective normal distributions.

11. The method according to claim 10, wherein further comprising displaying the assessed result.

12. The method according to claim 10, wherein the deriving of the ICRs of the bone segments includes:
overlapping the images of the bone in the flexion position and the extension position with each other and selecting at least two specific points corresponding to each other in the images of the bone;
connecting the at least two points of the images in the flexion position and the extension position by straight lines;
calculating bisected positions of the straight lines and drawing vertical lines from the calculated positions toward a lower bone segment direction; and
deciding intersections of the vertical lines as the ICRs.

13. The method according to claim 12, further comprising ensuring the correspondence between the two points while the coordinate of the corresponding image is automatically found, if a coordinate of a point measured at a position is computed in order to enable the corresponding two points in the images of the flexion position and the extension position to be matched with each other.

14. The method according to claim 10, wherein the deriving of the ICRs includes obtaining at least three ICRs and deriving arithmetic average coordinates thereof as the ICRs.

15. The method according to claim 10, wherein the establishing of the normal distribution database includes:
photographing a plurality of normal bones in the flexion position and the extension position and acquiring the digitalized images;
deriving the ICRs of the bone segments from the digitalized images in the flexion position and the extension position and representing the ICRs of the bone segments by coordinates; and
setting regions, in which the ICRs of the plurality of normal bones are concentrated, as the normal distributions and storing the regions in the normal distribution database.

16. The method according to claim 15, wherein the assessing of the kinematic status includes determining that the rotational bone kinematics is normal if the coordinates of the ICRs of the bone segments are in the respective normal distributions.
